(19) United States
(12) Patent Application Publication
(10) Pub. No.: US 2004/0067530 A1
(43) Pub. Date: Apr. 8, 2004

(54) Electronic sensing of biomolecular processes

(75) Inventor: George Gruner, Los Angeles, CA (US)

Correspondence Address: GATES & COOPER LLP, HOWARD HUGHES CENTER, 6701 CENTER DRIVE WEST, SUITE 1050, LOS ANGELES, CA, 90045, US

(73) Assignee: The Regents of the University of California

(21) Appl. No.: 10/431,963

(22) Filed: May 8, 2003

Related U.S. Application Data
(60) Provisional application No. 60/378,843, filed on May 8, 2002.

(51) Int. Cl. G01N 33/53

(52) U.S. Cl. 435/7.1

(57) ABSTRACT

The disclosure provided herein describes methods for the detection of conformational changes and/or interactions between biomolecules and molecules which bind to the biomolecules. The method is based on detection of alterations in resistance of a semiconductor nanostructured material coupled to a biomolecule where the resistance is modulated by the conformational state of biomolecules such that a change in the level of resistance so measured provides an indication of a change in the conformation of the biomolecule.

### CROSS REFERENCE TO RELATED APPLICATIONS

This application claims the benefit of U.S. provisional patent application serial No. 60/378,843, filed May 8, 2002. The entire content of this provisional patent application is incorporated herein by reference.

### FIELD OF THE INVENTION

The present invention provides methods for the detection, identification and/or quantification of biomolecules such as polynucleotides and polypeptides and to reagents and detector apparatus adapted for performing these methods.

### BACKGROUND OF THE INVENTION

The detection of conformational changes such as DNA-protein, protein- protein or protein-virus interactions is one of the important challenges of biotechnology, and detecting conformational changes and hybridization of biomolecules such as DNA, RNA also underlines many of the tools of biotechnology. Such detection at present is achieved by utilizing spectroscopic (such as fluorescence) and chemical (such as DNA sequence detection) methods. In particular, detecting hybridization of DNA segments in gene chips (see, e.g. M.Chee et al., Science 274, 610 (1996) and M.Shena et al., Proc. Natl. Acad. Sci. U.S.A. 93, 10614 (1996)) is of primary importance. At present utilizing fluorescent labeling of one strand of DNA performs this task. Hybridization leads to changes in the fluorescent intensity, which can be detected by a suitable optical arrangement. Fluorescent tagging of the probe or of the target introduces an additional step in sample preparation, and tagging may introduce modification of the DNA conformation, thus introducing errors in the analysis. In addition, photobleaching leads to a time limit of the analysis.

Alternative techniques proposed, such as using nanoparticle probes (see, e.g. T.A. Taton et al., Science 289, 1757 (2000); and J. Reichert et al., Anal. Chem 72, 6025 (2000)) also involve additional preparation steps, and the labels may influence duplex formation. Detection through nanomechanics is at an early stage of development (see, e.g. J.Fritz et al., 288, 316 (2000)). Electronic detection of biological molecules has been proposed and utilized earlier (see, e.g. U.S. Pat. Nos. 4,816,118, 3,957,612 and 5,001,531). Electronic detection has been demonstrated on functionalized carbon-based nanotubes (see, e.g. Yi. Cui et. al. Science 293, 1298 (2001)).

Currently there is a need in the art for additional methods and devices that overcome technical limitations associated with the existing technology. The methods and devices disclosed herein satisfy this need.

### SUMMARY OF THE INVENTION

The disclosure provided herein describes methods for the electronic monitoring of biomolecules in order to observe conformational changes and/or ligand binding. The methods disclosed herein employ semiconductor nanostructured materials such as nano-structured chemical sensors (see, e.g. Solid State Gas Sensors Eds. P.T.Moseley and B.C.Tofield (Adam Hilger, Bristol 1987)). Typical nano-structured sensors are sensors where the sensing element consists of a material with structural characteristics on the length scale of less that 100 nm. An illustrative example of such sensing elements are nano-particles of an oxide semiconductor or a non-porous material. Another illustrative example is a nanostructured material coated with a layer sensitive to an analyte. The structural attributes of such sensing elements leads to a large surface area which is available for immobilization of a biomaterial which then facilitates their use in methods for the electronic monitoring of biomolecules. The detection of sensing involves the transfer of a signal between two nanostructured materials in contact with each other, this signal can be electronic or mechanical. In this context, elements such as nano-structured chemical sensors can be used as biosensors by exploiting their surface layer as a medium for attachment of biomolecules as well as for the evaluation of the status of these biomolecules by monitoring the resistance across the material.

### BRIEF DESCRIPTION OF THE FIGURES

FIG. 1A shows the schematic outlay of the semiconductor sensor. An applied voltage leads to a current which is monitored. FIG. 1B shows semiconductor nano-particles with their surface layer. The arrow indicates the electronic conduction path. FIG. 1C shows a semiconductor nanoparticle coated with another material, with the arrows indicating the electronic conduction path.

FIG. 2 is a schematic representation of the functionalized nano-structured sensor-material, with a target bio-material also shown.

FIG. 3 provides a schematic of the nanotube field effect transistor (NTFET) that uses a network of nanoparticles as conducting channel. A polymeric functional layer, which coats the network, functionalized with a molecular receptor, a protein that recognizes a biomolecule (not shown) can be incorporated into the structure. S; source, D; drain, G: gate.

(Dimensions are not in scale.)

FIG. 4 provides an illustration of a biotinylation reaction of the polymer layer (PEI and PEG).

FIG. 5 provides an AFM image of the polymer-coated and biotinylated NTFET after exposure to streptavidin labeled with gold nanoparticles

(10 nm diameter).

FIG. 6 provides gate voltage dependence of the source-drain current Isd of a typical device before and after PEI/PEG polymer coating and after biotin attachment to the polymer layer.

FIG. 7 provides change of the device characteristic Isd(Vg) upon exposure to streptavidin. Different devices with similar characteristic as observed before polymer coating were used for a, b, and c. (a) Gate voltage dependence Isd of the biotinylated, polymer-coated NTFET in the absence and in the presence of streptavidin. (b) Current-voltage dependence Isd(Vg) of the bare NTFET device to nonspecific protein binding. (c) Isd(Vg) of the polymer-coated NTFET device in the absence and presence of streptavidin. (d) Isd(Vg) of the biotinylated, polymer coated NTFET device in the absence and presence of streptavidin that was preincubated with biotin.

FIG. 8 provides a graph of data in experiments examining the affinity of proteins to nanotubes using multiwall nanotubes. Nanotubes (2 mg) dispersed in were incubated with BSA-dye conjugate (1 mg/ml, 1.25 microgram) overnight, were washed with PBS. Depletion of BSA from the solution was evaluated using UV absorption (580 nm) of fluorescent dye. As shown in this graph, when adding proteins (BSA) the resistance drops. This is due to a combination of factors: due to the conducting channel provided by the buffer and due to the change of the resistance of the network due to protein attachment. When appropriate correction for the buffer resistance is made, the resulting network resistance (considering the film to be in parallel with buffer) it is approximately 30% higher than the value of the resistance in just buffer. This graph also illustrates the resistance of network device in air, and after application of buffer and BSA. It is apparent that the network resistance measured after buffer removal is higher than before the protein attachment.

FIG. 9 provides a graph of data from a simple analysis made where the values of the network's resistance are assumed to correspond to the values measured on the “wet” section of the graph.

FIGS. 10A-10D provides a graph of data from experiments as described in Example 2 below showing film resistance under various conditions with only buffer or buffer+protein is applied.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The disclosure provided herein describes a nano-structured sensors where the detection involves two nanostructured materials in contact, with the detection involving the transmission if a signal between the two nano-structured elements. A typical sensor can be a chemical sensor (see, e.g. Solid State Gas Sensors Eds. P.T.Moseley and B.C.Tofield (Adam Hilger, Bristol 1987)), or as a biosensor by exploiting the surface layer both as a medium for attachment of biomolecules and detection through the resistance across the material, or across another material in contact with the surface layer. Typical nano-structured sensors are sensors where the sensing element consists of a material where at least one of the dimensions of the material has the length of less that 100 nm. One representative example of such a sensing element are nano-particles of an oxide semiconductor or a non-porous material. This structural attribute of this sensing element leads to a large surface area which is available for immobilization of a biomaterial. Consequently, such semiconductor nanostructured materials can be utilized for the electronic monitoring of biomolecules which undergo conformational changes and/or ligand binding. Another example is a nanostructured material on which another material, such as a polymer film or lipid bilayer is deposited.

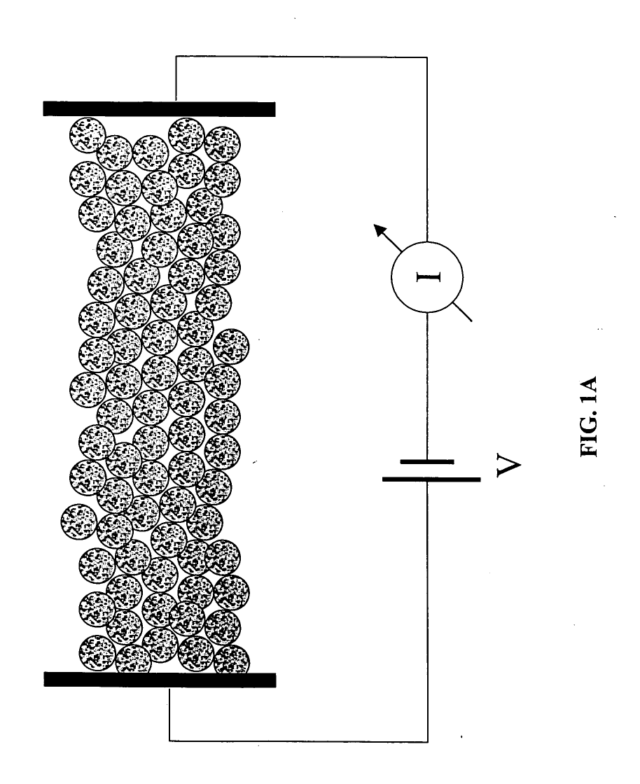

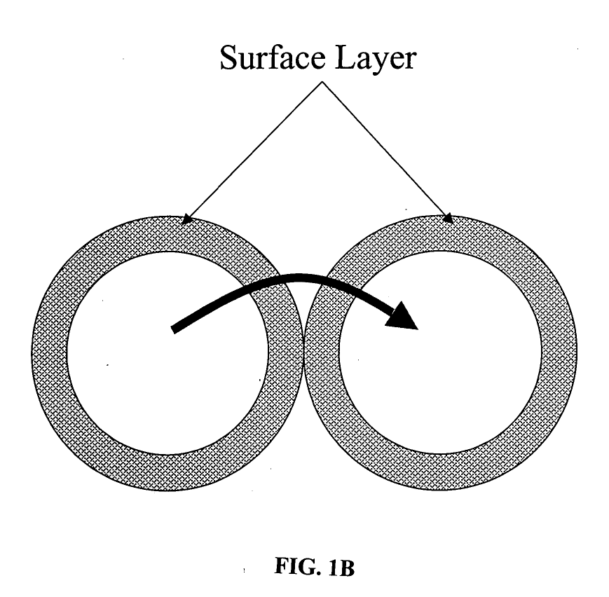

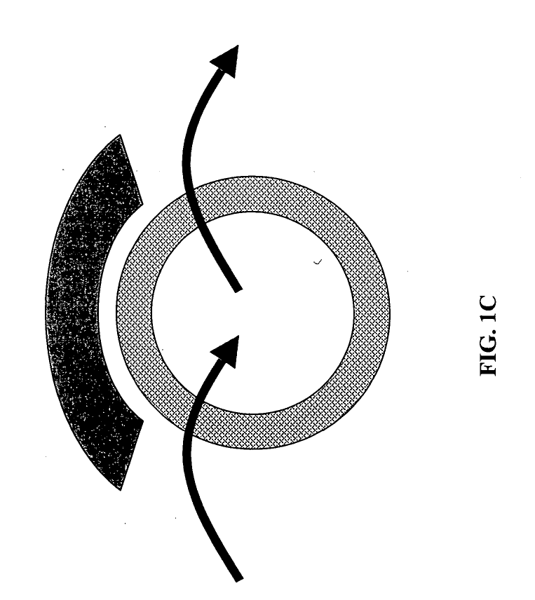

Such materials have been employed earlier as a chemical sensor. Among the various semiconductor nanostructured sensors oxide semiconductors, such as SnO₂ are the most widely used nano-porous material used for sensing of chemical elements (see, e.g. S.Trautweiler et al.; New Silicon based metal-Oxide Chemical sensors www.sensormag.com/articles/0999). The sensing is through the resistance, which changes upon the adsorption of the species on the material. The operation of the oxide semiconductor based chemical sensor is as follows. The oxide semiconductor is n-type, having an excess electrons, this achieved by doping. Due to the n-type character of the carriers, oxygen molecules adsorbed at the surface dissociate creating O (minus) species and thus an equilibrium charged surface layer, which in general is called the depletion layer. The surface of the material can also be functionalized by depositing an appropriate material on the surface, such functionalization routes are well established for various oxide materials. Other species, such as DNA oligomers or proteins which may attach to the immobilized biomaterial lead to changes in the overall electron concentration at the surface—which is detected through the change of the resistance measured across the (semiconducting) SnO₂ film, deposited onto an appropriate surface. Extraordinary detection sensitivity can be achieved, because the transport of electronic charges across the surface layers is extremely sensitive to charge or dipole induced modifications of the layer. The device is displayed in FIG. 1A, with the structural elements —nano-particles across which electronic conduction occurs—displayed on FIG. 1B. The arrow indicates the path of electronic conduction across the depletion layer. In another embodiment, a layer, sensitive to the analyte can be deposited on the nanostructured material, and transduction of a signal from the deposited material to the nanostructured material leads to the change of the resistance of the nanostructured material.

The detection of conformational changes, interaction between proteins and DNA duplex formation is achieved as follows. On the surface of the sensor a biomolecule (the probe) is deposited and immobilized using well know techniques for biomaterial immobilization. Depending on the use, a typical biomaterial can be a molecules such as a: a single stranded DNA oligonucleotide; an RNA; an antibody to the protein one intends to detect; and any one of a wide variety of polynucleotides and polypeptides known to interact with binding partner such as a complimentary polynucleotide, a ligand (including small molecules) etc. In this context, the introduction of complimentary DNA, or antigen which binds to the antibody or polypeptides that interact with etc., the binding partner changes of the surface characteristics leads to a change of an electronic signal between the nano-particles, which is detected. Artisans understand that devices, methods and materials of the invention can be used to examine the interaction of both biomolecules as well as analogously interacting chemical species.

Another configuration in embodiments of the invention includes the nanostructured material on which another material is deposited. For example, biomolecules can be immobilized on the surface of the deposited material. Conformational changes or ligand binding can then be detected by transducing an electronic or mechanical signal—that arises as the consequence of the conformational change, duplex formation, antibody-antigen or ligand binding—in the deposited material to the nanostructured material. Other materials include polymers that prevent non-specific bio-molecule binding and a layer of molecules on which bio-molecules ate attached.

Illustrative Embodiments of the Invention

Device Elements

Preferred embodiments of the invention include a nanostructured material network on a substrate. Preferable network properties include an interconnected network so that the current (or most of the current) flows across the network. Preferable network properties further include a “loose” network so that the elements of the network in contact with the environment. Network density optimization can be important and is probably close to a “percolation threshold”. Preferable elements for use with embodiments of the invention include nanowires, nanotubes, and nanoparticles, such as metal oxides. Preferable elements for use with embodiments of the invention further include electronic leads to the network (e.g. a gate arrangement for transistor operation).

Preferred embodiments of the invention can also include a layer for bio-functionality, typically recognition molecules (e.g. single strand oligomer, antibody etc.) attached to the network, a coating to prevent false positives, a coating including recognition molecules and/or a coating to which recognition molecules, antibodies are attached (e.g. an electronic lead arrangement for buffer conductance compensation).

In certain embodiments of the invention include compensation for buffer conductivity (e.g. same arrangement as above but without the nanoparticle network).

Detection Methods

In various embodiments of the invention, detection methods include: dc voltage or current; ac voltage and current; electrical pulses; and/or electrical fluctuations due to the interactions between biomolecules (e.g. proteins/DNA). Embodiments of the invention include bio-molecule detection: (1) in buffer, e.g. for real time monitoring the change of resistance; and/or (2) after buffer removal by detecting the attachment or presence of biomolecule by measuring the change of resistance with and without the target molecule.

Methods for Making Embodiments of the Invention

In preferred embodiments of the invention a first step in the functionalization of the surface is to attach a molecule or reactive group which provides a common link for a number of subsequent, different, specific functionalizations. For example, biotin can be coupled to semiconductor surfaces through known methods (see, e.g. A. N. Asanov et al, Anal. Chem. 70, 1156 (1998). In such embodiments, after, biotinylated oligonucleotides, biotinylated secondary antibodies, etc., can be coupled through a streptavidin bridge. Alternatively, probe molecules, e.g. DNA oligonucleotides, can be directly adsorbed on the surface (see, e.g. P.M Armistead and H. Holden Thorp, Anal. Chem. 73, 558 (2001); adsorbing a secondary antibody would provide binding sites to any specific primary antibody. By electronically detecting ligand binding, an array of such μm scale sensors can allow thousands of binding assays to be run in parallel with very small quantities of material.

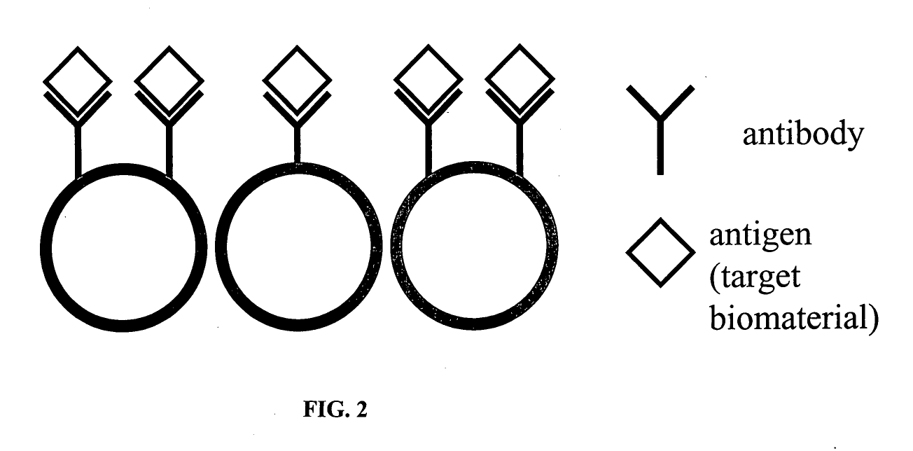

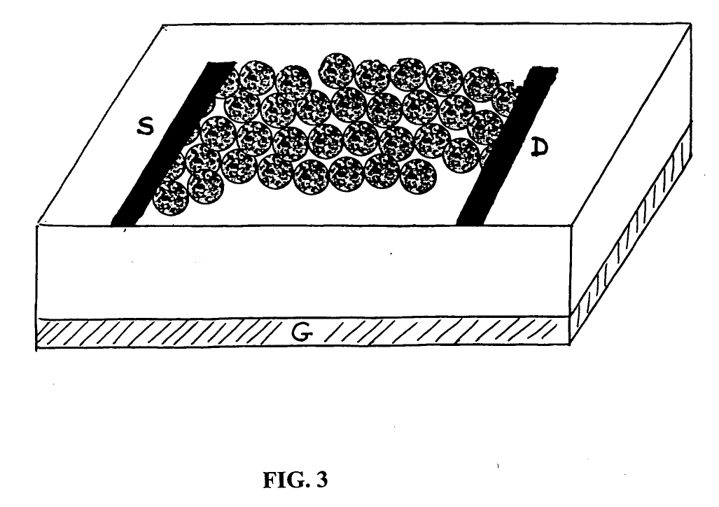

Such functionalized nano-structured material is shown in FIG. 2. The resistance of the nano-structured sensor material is monitored and the resistance value, with the biomaterial immobilized on the surface can be measured. The target molecules attach specifically to sensor attached biochemical receptor, this attachment resulting in a change of the charges at the surface layer of the nano-structured material. This change therefore leads to the change of the resistance of the device, or other electronic characteristics associated with the motion of the electrons across the device. Such change has been established in case of chemical species, and forms the basis of chemical sensor application of these materials. With the resistance monitored during the application of a bioassay, the change of the electronic signal leads to the detection of the conformational change, such as duplex formation or protein attachment to the immobilized biomaterial.

While the device is preferably used in a bioassay environment, but a simple construction can be made to detect biomaterials which exists in a gaseous or air environment (e.g. to sense biohazards such as smallpox and anthrax etc., toxic gases such as VX, sarin, etc). In this case a device will typically consist of the elements comprising: an air flow system which takes samples of the gaseous or air environment; a mechanism which allows the sampled gas or air to dissolved in a bioassay; and a mechanism, such as used in a “lab on a chip” arrangement which flows the bioassay over the detector.

Illustrative Nanoparticle Network Fabrication

In preferred embodiments of the invention, nanotube networks will initially be fabricated by direct deposition of single walled nanotubes on a silicon surface. While silicon is a preferred surface, artisans understand that deposition to other surfaces is also contemplated. The networks can be used to fabricate transistor devices, with transconductances close to that obtained for an array of nanotubes with conducting and semiconducting nanotubes involved.

A preferred example of the fabrication of such networks involves nanotube films. We have developed a method of laying down nanotube films employing porous alumina membranes as filters and measurement substrates. Such alumina membranes are an ideal substrate as they can be made optically flat and are easily characterized as a background material. The method has a number of important advantages over simple air drying of a liquid suspension for carbon nanotubes. The weak residual interaction between nanotubes in solution results in large flocculation effects (clumping) as the suspension dries. Air-drying of such a suspension on sapphire results in totally unsuitable results, where nanotubes form 0.1-mm ‘piles’ upon drying. Filtration through an inert substrate such as alumina allows liquid to be removed before large-scale structures form in the suspension. In this method, nanotubes are ultrasonically dispersed in spectroscopic grade dichlorobenzene or xylene. Then this suspension is deposited onto vacuum pumped alumina membranes (0.2 micron pore size) where the liquid can be removed on a time scale short enough to not allow flocculation to occur. In addition, we have recently developed a method whereby these deposited films can be subsequently floated on top of a water/isopropanol solution and then redeposited on a arbitrary substrate (silicon or sapphire). This method resembles the Langmuir-Blodgett film deposition that is used to create thin organic monolayer films.

The films consist of an interconnected network of bundles of single wall nanotubes, with a typical tubule diameter of 10 nm—comparable to a typical multiwall nanotube, one which significant protein attachment was found (see below).

Illustrative Electronic, Source-Drain Contact Fabrication

In preferred embodiments of the invention, the sensor architecture includes a nanotube network with two contact electrodes attached, and combined with a simple fluidic cell. 1000 Angstroms of titanium is deposited through a mask, providing electrodes on the coverslip and film. Wires are then attached directly to the titanium which is in contact with the network using silver epoxy.

Buffers

Due to the finite conductivity of the buffer the device in a buffer environment can be represented as two parallel conducting channels, with both channels, in principle changing during the experiment.

The resistance of a SWNT network strongly depends on the density of the network. The objective is then to maximize to buffer's resistance while minimizing the network's resistance, but keeping in mind that the buffer's resistance has to be large enough (i.e. have enough salt) so that the protein finds itself in a biological environment.

Protein solutions where the protein concentration and buffer concentration spanned 6 orders of magnitude each were used with solutions of varying molecular weight and protein size.

In the context of the embodiments of the invention disclosed herein, artisans will understand that the term “nanoparticle” includes bulk nanoparticles, such as oxide nanoparticles, cocoons, nanowires, nanofibres, nanotubes, bundles of nanotubes, fullerenes and the like.

In the context of the embodiments of the invention disclosed herein, artisans will understand that the term “network” comprises a collection of nanoparticles as defined above, providing a conduction path between two electrodes. The conducting path dominantly includes the nanoparticles in close proximity to each other, with the current flowing from one nanoparticle to the other, to the next, etc. In certain embodiments of the invention, networks are preferred instead of a film due to their ability to: (1) provide a higher surface area; (2) conducting part more sensitive to environment and to attached/detected biomolecules; and (3) exhibit a size compatibility with proteins. In addition, a network, in contrast to individual nanoscale interconnects provides robustness, fault tolerance, and reproducibility while preserving sensitivity. Networks are distinguishable from a porous material.

In the context of the embodiments of the invention disclosed herein, artisans will understand that the term “semiconducting” simply means providing conduction. For example in embodiments of the invention, a network can include both semiconducting and metallic nanoparticles.

Embodiments of the invention disclosed herein can include a recognition layer. Typically this includes a polymer with protein-repelling properties, such as PEG, and others known in the art. Optionally this layer has self assembly properties. In one embodiment of the invention, this layer is a dense set of antibodies.

Embodiments of the invention disclosed herein include a substrate, preferably a silicon or a polymeric substrate. Alternative substrate materials such as glass, metal, plastic and the like are contemplated. Embodiments of the invention disclosed herein further include electronic interconnects, for example in resistor (source and drain) and/or transistor (source and drain together with gate) configuration.

Advantage Over Prior Art

Devices known in the art include electronic devices where the sensing element is a continuous film and electronic devices with one nanoparticle element such as a nanowire or a nanotube. The disclosed architecture of the embodiments of the invention have several advantages over existing devices. A first such advantage is a simplicity in fabrication. In addition, there is no need for patterned catalyst, and may be for a structure where nanoparticles are present in one location and not present in others on the wafer. Yet another advantage is a large surface area available for immobilization. Yet another advantage is a selected quasi one-dimensional conduction path. Yet another advantage is a size compatibility with proteins allowing protein selective immobilization. Yet another advantage is that as many nanoparticles act as the conducting element, statistical averaging will occur, strongly reducing the signal variation from device to device. Yet another advantage is that the signal is a simple dc resistance (or dc voltage), eliminating the need for an elaborate drive and detection electronics. Yet another advantage is that by virtue of the large number of nanoparticles involved, the structure is also “defect tolerant”.

### EXAMPLES

### Example 1

### Electrical Detection of Specific Protein Binding Using Nanotube FET Devices

This Example appears in publication: Star et al., NANO LETTERS 3(4): 459-463 (2003). The example involves one nanotube as sensing element but the art can be equally well applied to a collection of nanoparticles, in particular to nanotube networks of to networks of nanofibres, or any other nanostructured material onto which the polymers mentioned in the Example can be deposited.

In this example we used nanoscale field effect transistor devices with carbon nanotubes as the conducting channel to detect protein binding. A PEI/PEG polymer coating layer has been employed to avoid nonspecific binding, with attachment of biotin to the layer for specific molecular recognition. Biotin-streptavidin binding has been detected by changes in the device characteristic. Nonspecific binding was observed in devices without the polymer coating, while no binding was found for polymer-coated but not biotinylated devices. Streptavidin, in which the biotin-binding sites were blocked by reaction with excess biotin, produced essentially no change in device characteristic of the biotinylated polymer25 coated devices.

Current biological sensing techniques commonly rely on optical detection principles that are inherently complex, requiring multiple steps between the actual engagement of the analyte and the generation of a signal, multiple reagents, preparative steps, signal amplification, complex data analysis, and relatively large sample size. The techniques are highly sensitive and specific but more difficult to miniaturize. Electronic detection techniques may offer an alternative, but their potential has not yet been explored fully. Field effect transistors (FETs) fabricated using semiconducting single wall carbon nanotubes (nanotube FETs, NTFETs) have been extensively studied (1,2). Such devices have been found to be sensitive to various gases, such as oxygen and ammonia, and thus can operate as sensitive chemical sensors. The mechanism responsible for the change of device characteristic is thought to be a charge-transfer reaction between the analytes and the nanotube. NTFET devices (3,4) together with devices based on nanowires (5), are also promising candidates for electronic detection of biological species. Various groups have examined the conformational compatibility-driven by size issues as well as hydrophobic effects-between proteins and carbon nanotubes using streptavidin, and found that the protein is able to crystallize in a helical conformation around multiwall carbon nanotubes (6). We have also shown (7) that functionalization of the nanotubes with carboxylic groups, thereby rendering them more hydrophilic, does not lead to protein attachment, thus opening up the avenues for specificity. Researchers have also made (3c,4) some attempts at functionalizing single-wall carbon nanotubes to make them biocompatible, capable of recognizing proteins by using noncovalent binding between a bifunctional molecule and the nanotube to anchor a bioreceptor molecule with a high degree of control and specificity.

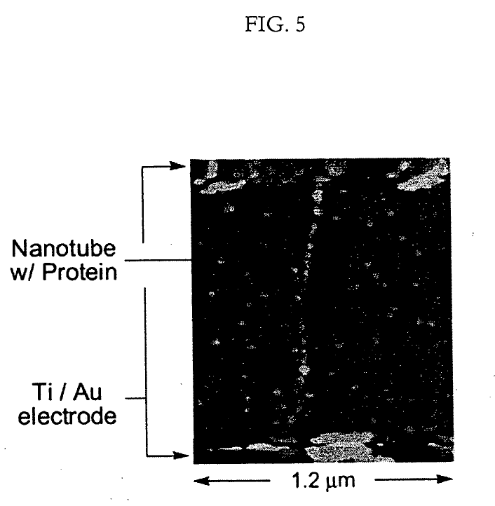

In this example we report taking these advances one step further, by using a sensor architecture that allows the detection of protein-receptor interactions (using biotin-streptavidin binding as an example) and, at the same time, reduces or eliminates nonspecific protein binding. FIG. 5 schematically depicts a sensor architecture that uses a NTFET as a transducer; it is covered with a polymer coating that has hydrophilic properties and onto which biotin is attached. Polymer functionalization in this sensor architecture has several advantages. First, the polymer is used to attach molecular receptor molecules to the sidewalls of nanotubes. Several examples of covalent chemical attachment of biological molecules to nanotubes, including proteins and DNA, have been recently published (8). Covalent modification, however, has the disadvantage that it impairs physical properties of carbon nanotubes. For these reasons we have employed a supramolecular approach, namely, the noncovalent functionalization of carbon nanotubes by employing polymer coatings (9). Second, polymer coatings have been shown to modify the characteristics of nanotube FET devices, and thus the coating process can be readily monitored. In particular, coating NTFETs with polyethylene imine (PEI) polymer was found (10) to shift the device characteristic from p- to n-type, presumably due to the electron-donating ability of amine groups in the polymer. Third, the polymer coating could be used to prevent nonspecific binding of proteins. A variety of polymer coatings and self-assembled monolayers have been used to prevent binding of undesired species on surfaces for biosensor and biomedical device applications (11). Among the various available polymers for coating, poly(ethylene glycol) (PEG) is one of the most effective and widely used. This layer, due to its hydrophilicity, reduces the affinity of nanotubes toward protein binding.

We have chosen the biotin-streptavidin binding to demonstrate the effectiveness of the device architecture. This binding serves as a model system for protein interactions (12) has been extensively studied, and the binding is well understood. In our procedure, after incubation, the device was washed and dried, and the device characteristics were examined after drying. While we have explored the device response in a buffer, our objective here is to examine the changes of the device characteristic, brought about by the different chemical and biological modifications on the electronic response, such direct correspondence being somewhat obscured in a buffer environment (13).

FET devices with nanotubes as the conducting channel were fabricated using nanotubes grown by chemical vapor deposition (CVD) on 200 nm of silicon dioxide on doped silicon from iron nanoparticles with methane/hydrogen gas mixture at 900° C.; electrical leads were patterned on top of the nanotubes from titanium films 35 nm thick capped with gold layers 5 nm thick, with a gap of 0.75 Im between source and drain. Multiple nanotubes connected the source and drain electrodes, with the individual tubes varying from metallic to semiconducting (14). Consequently, a range of device modulations (expressed as the ratio of the “on” to the “off” source-drain current, measured at −10 V and +10 V gate voltage, respectively) were observed. The devices displayed p-type transistor behavior, as has also been observed by others (1,2). In this example we have examined the dependence of the source-drain current, Isd, as function of the gate voltage Vg, Isd(Vg), measured from +10 V to −10 V, and we refer to this response as the “device characteristic”. After conducting initial electrical measurements to establish the device characteristic, the substrates were submerged in a 10 wt % solution of poly(ethylene imine) (PEI, average molecular weight _{—}25 000, Aldrich) and poly(ethylene glycol) (PEG, average molecular weight 10 000, Aldrich) in water overnight, followed by thorough rinsing with water. Commercial polyethyleneimine (PEI) was used; this form is highly branched, has a molecular weight of about 25 000, and contains about 500 monomer residues. About 25% of the amino groups of PEI are primary with about 50% secondary, and 25% tertiary. A thin layer (<10 nm) of polymer material coated the devices, as observed by atomic force microscopy.

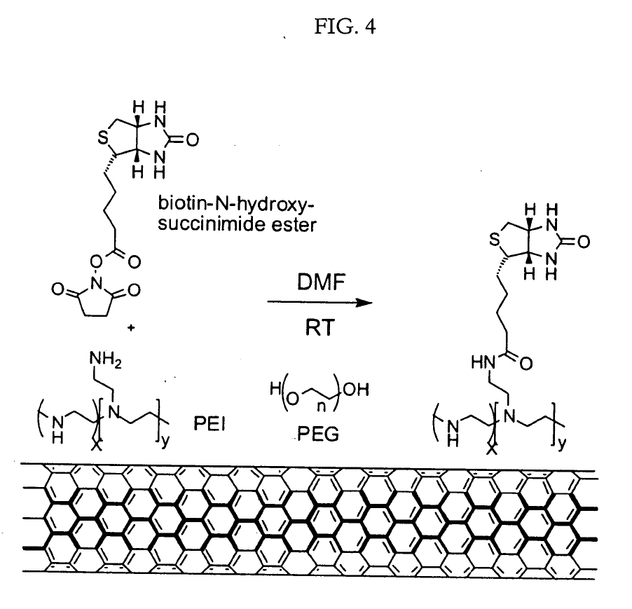

The polymer-coated devices were biotinylated by submerging them in a 15 mM DMF solution of biotin-N-hydroxysuccinimide ester (Sigma) at room temperature. This compound readily reacts with primary amines in PEI under ambient conditions, leading to changes of the device characteristic as will be discussed below. After soaking overnight, devices were removed, rinsed with DMF and deionized water, blown dry in nitrogen flow, and dried in a vacuum. FIG. 4 depicts the scheme by which biotin was attached to the polymer coating. The biotinylated polymer-coated devices were exposed to the 2.5 IM solution of streptavidin 15 in 0.01 M phosphate buffered saline (pH ) 7.2, Sigma) at room temperature for 15 min. Subsequently, the devices were thoroughly rinsed with deionized water and blown dry with nitrogen. Several control experiments have also been performed in order to demonstrate the effectiveness of the polymer layer in the prevention of nonspecific binding.

An atomic force microscope (AFM) image of one of the devices after exposure to streptavidin labeled with gold nanoparticles is shown in FIG. 5. Light dots represent gold nanoparticles (10 nm), and thus indicate the presence of streptavidin. Based on the image, we conclude that streptavidin is effectively attached to the nanotubes, due to the strong adsorption of the PEI polymer to the sidewalls of the nanotubes, which was biotinylated after deposition. With a nanotube length of 800 nm and a gold sphere diameter of 10 nm, it is expected that, upon full coating, there are approximately 80 streptavidin molecules in direct interaction with the nanotube conducting channel. (This assumes that, on the average, one streptavidin molecule per gold nanoparticle is attached to the nanotube.)

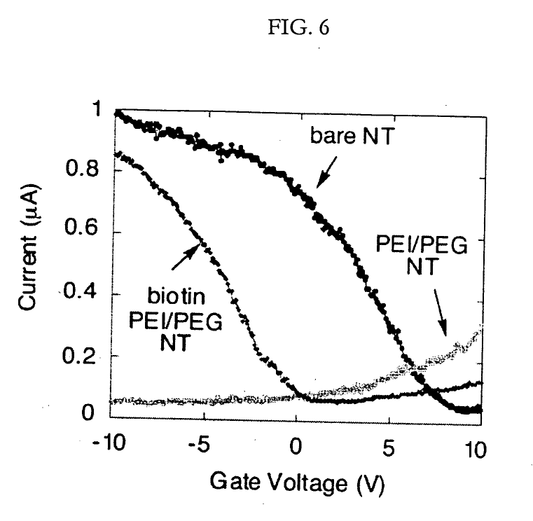

Next we discuss the change of the device characteristic in response to the steps we have taken. The device characteristic before chemical modification is p-type, in an ambient environment, presumably due to exposure to oxygen (16). Coating the device with the mixture of PEI and PEG polymers results in an n-type device characteristic (FIG. 6). This effect, which has been observed (10) before, probably results from the electron donating property of the NH2 groups of the polymer. The electronic characteristic of the device after 18 h of biotinylation reaction is also depicted in FIG. 6. Attachment of biotin is through covalent binding to the primary NH2 group, thereby reducing the overall electron donating function of PEI and leading to a device characteristic that is consistent with removal of electrons from the device. As only the primary NH2 sites are involved in binding to biotin, the p-type conductance observed before coating is not fully recovered (17).

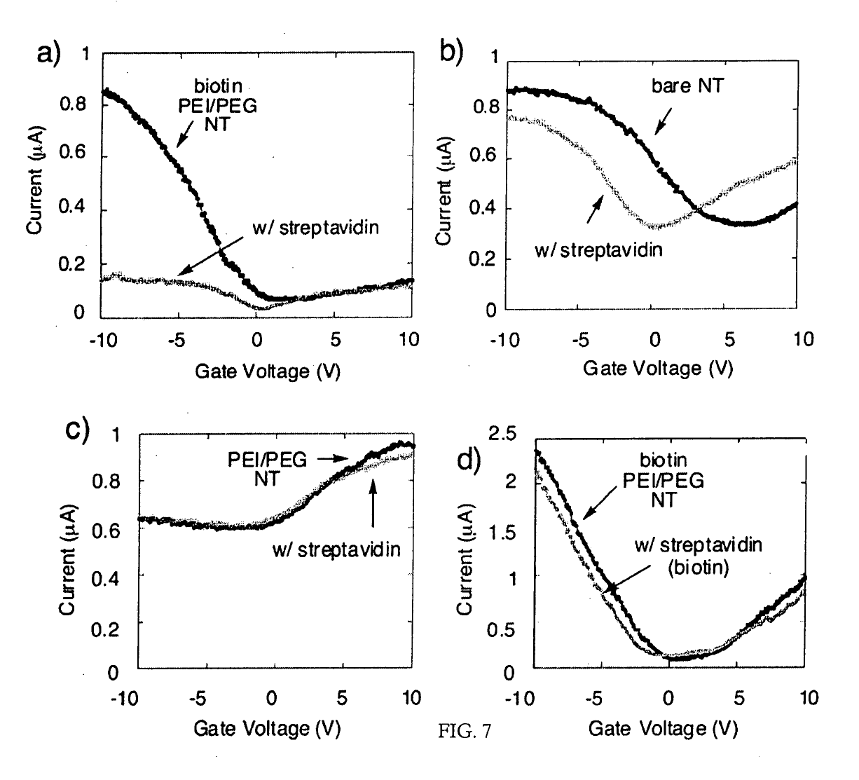

The effect of exposing the biotinylated polymer-coated device to a streptavidin solution and the control experiments (conducted on different devices) are shown in FIG. 7. FIG. 7a shows a striking loss of source-drain current for negative gate voltages after exposure to streptavidin and consequent streptavidin-biotin binding with little evidence for the shift of the device characteristic toward negative or positive gate voltage. Several control experiments were performed to demonstrate the effectiveness of the device architecture in avoiding false positives and in detecting specific protein binding. First, we have exposed the uncoated NTFET device to streptavidin and have found a change of the device characteristic, as shown in FIG. 7b, indicating attachment of streptavidin to the device. Note, however, that in this case the primary effect is the shift of the device characteristic toward negative gate voltage. In contrast, when the device was polymer-coated, but not biotinylated, no changes occurred upon exposure to streptavidin (FIG. 7c). This suggests the effectiveness of the polymer coating in preventing direct, nonspecific interaction of streptavidin with the nanotube. Finally, addition of a streptavidin in which the biotin-binding sites were blocked by complexation with excess biotin produced essentially no change in device characteristic of the biotinylated polymer-coated device (FIG. 7d).

Several conclusions on the effect of biomolecules on the device electronics can be drawn. First, exposing the bate, uncoated device to streptavidin leads to the shift of the transconductance toward negative gate voltages, thereby rendering the device less p-type, with little reduction in the magnitude of the transconductance. This indicates that the primary effect of the nanotube-streptavidin binding is a charge-transfer reaction with streptavidin donating electrons to the nanotube (16). Biotin-streptavidin binding has a different effect; in this case the Isd is reduced. Without being bound by a specific theory, we suggest that upon streptavidin-biotin binding, geometric deformations occur, leading to scattering sites on the nanotube, and thus to reduced conductance. At the same time the device characteristic is modified only for negative gate voltages (see FIG. 7a), leaving the transconductance in the positive gate voltage region unaffected. We have observed (17) similar features in devices to which charge carriers were deposited, and we have argued that the observation is due to localization (delocalization) of positively (negatively) charged ionic entities by a negatively (positively) charged surface. Such a mechanism may also be effective here, and the mechanism may open the way for electronic modification of bioreactions.

With improvements in NTFET devices, they may also be rendered sensitive enough that single protein detection and monitoring can be achieved. As can be inferred from FIG. 7a, the total change in transconductance exceeds the noise level by a factor of 10. According to an AFM image of the device (FIG. 5), there are about 100 gold nanoparticles, and approximately 100 protein molecules (assuming one protein per gold nanoparticle binding to the tube) in close proximity to the carbon nanotube. Combining these two numbers, our current detection level can be estimated to be of the order of 10 streptavidin molecules. Similar detection sensitivity can be inferred from experiments we have conducted on uncoated nanotubes incubated with streptavidin (FIG. 7b). This is in contrast to the relatively modest change observed in devices where the active element is a nanowire5—a channel with a substantially larger cross section.

Electronic sensing using devices with nanotubes as the conducting channel offers several advantages. Such sensors are small and fast, and the active detection area is sized for individual proteins or viruses. These sensors are extremely sensitive, as all the current passes through the detection point. Most importantly, at a later stage the devices can be made specific to individual molecules; potentially their response to different species can be varied in a controlled way using chemical and biological functionalization. These concepts could conceivably be extended at a later stage to include cell-based electronic sensing (measuring the electronic response of living systems) and to using nanoscale devices for in-vivo applications (studying cell physiology, medical screening and diagnosis). The devices can be turned into devices where, by applying a voltage between elements of the sensor, surface charges can be created on the sensing element where the bio-molecules are immobilized. Such surface charges will interact with the charged biomolecules affecting biological function.

### REFERENCES

(1) Bachtold, A.; Hadley, P.; Nakanishi, T.; Dekker, C. Science 2001, 294, 1317-1320.

(2) Martel, R.; Schmidt, T.; Shea, H. R.; Hertel, T.; Avouris, Ph. Appl. Phys. Lett. 1998, 73, 2447.

(3) (a) Collins, P. G.; Bradley, K.; Ishigami, M.; Zettl, A. Science 2000, 287, 1801. (b) Kong, J.; Franklin, N. R.; Zhou, C.; Chapline, M. G.; Peng, S.; Cho, K.; Dai, H. Science 2000, 287, 622. (c) Chen, R. J.; Zhang, Y.; Wang, D.; Dai, H. J. Am. Chem Soc. 2001, 123, 3838-3839.

(4) Shim, M.; Kam, N. W. S.; Chen, R. J.; Li, Y.; Dai, H. Nano Lett. 2002, 2, 285-288.

(5) Cui, Y.; Wei, Q.; Park, H.; Lieber, C. M. Science 2001, 293, 1289-1292.

(6) Balavoine, F.; Schultz, P.; Richard, C.; Mallouh, V.; Ebbesen, T. W.; Mioskowski, C. Angew. Chem., Int. Ed. Engl. 1999, 38, 1912-1915.

(7) Gruner, G.; Gabriel, J.-C.; Zocchi, G., unpublished.

(8) (a) Baker, S. E.; Cai, W.; Lasseter, T. L.; Weidkamp, K. P.; Hamers, R. J. Nano Lett. 2002, 2, 1413-1417. (b) Huang, W.; Taylor, S.; Fu, K.; Lin, Y.; Zhang, D.; Hanks, T. W.; Rao, A. M.; Sun, Y.-P. Nano Lett. 2002, 2, 311-314.

(9) (a) Star, A.; Stoddart, J. F.; Steuerman, D.; Diehl, M.; Boukai, A.; Wong, E. W.; Yang, X.; Chung, S. W.; Choi, H.; Heath, J. R. Angew. Chem., Int. Ed. 2001, 40, 1721-1725. (b) O'Connell, M. J.; Boul, P.; Ericson, L. M.; Huffman, C.; Wang, Y. H.; Haroz, E.; Kuper, C.; Tour, J.; Ausman, K. D.; Smalley, R. E. Chem. Phys. Lett. 2001, 342, 265-271. (c) Star, A.; Steuerman, D. W.; Heath, J. R.; Stoddart, J. F. Angew. Chem., Int. Ed. 2002, 41, 2508-2512.

(10) Shim, M.; Javey, A.; Kam, N. W. S.; Dai, H. J. J. Am. Chem. Soc. 2001, 123, 11512-11513.

(11) Ostuni, E.; Chapman, R. G.; Holmlin, R. E.; Takayama, S.; Whitesides, G. M. Langmuir 2001, 17, 5605-5620, and references therein.

(12) (a) Miyamoto, S.; Kollman, P. A. Proteins Struct. Funct. Genet. 1993, 16, 226-245. (b) Vajda, S.; Weng, Z.; Rosenfeld, R.; DeLisi, C. Biochemistry 1994, 33, 13977-13988.

(13) Star, A.; Han, T. R.; Gabriel, J.-C.; Bradley, K.; Gruner, G. “Electronic Detection in Liquids Using Nanotube FET Devices”, submitted for publication.

(14) NTFETs were fabricated using nanotubes grown by chemical vapor deposition, directly on 4″ silicon wafers (with 200 nm films of thermal SiO2) using a home-built apparatus. In a typical experiment, the wafer is covered with patterned photoresist and is spin coated with growth promoter containing nanoparticles of iron encased within a mesoporous material [(a) Li, W. Z.; Xie, S. S.; Qian, L. X.; Chang, B. H.; Zou, B. S.; Zhou, W. Y.; Zhao, R. A.; Wang G. Science 1996, 274, 1701-1703, (b) Pan, Z. W.; Xie, S. S.; Chang, B. H.; Wang, C. Y.; Lu, L.; Liu, W.; Zhou, W. Y.; Li, W. Z.; Qian, L. X. Nature 1998, 394, 631-632]. After liftoff in acetone, small patterned areas of the growth promoter are left on the wafer. The wafer is then introduced in a 5 in. tubular oven and treated at 900° C. in a methane and hydrogen flow for 15 min, allowing for the growth of SWCNT of 5-10 Im long and 1.5 to 3 nm in diameter. The hydrogen helps to prevent the deposition of amorphous carbon around the nanotubes as well as on the surface of the silicon [Ivanov V.; Nagy, J. B.; Lambin, Ph.; Lucas, A.; Zhang, X. B.; Zhang, X. F.; Bernaerts, D.; Van Tendeloo, G.; Amelinckx, S.; Van Landuyt, J. Chem. Phys. Lett. 1994, 223, 329-335]. Standard optical lithography and metal deposition are used to form the metal contacts on top of the grown nanotubes. A fair proportion of the tens of thousands of devices made on each wafer are p-type FETs with a modulation of 1, indicating that only semiconducting nanotubes are present. The devices used in this study were selected from among those. An AFM image of a typical device is presented in Supporting Information.

(15) Streptavidin is labeled with gold nanoparticles for the purpose of AFM imaging. Streptavidin (from Streptomyces aVidinii, Sigma Chemicals) without gold labeling had similar effect on the device characteristic but could not be detected by AFM.

(16) (a) Jhi, S.-H.; Louie, S. G.; Cohen, M. L. Phys. ReV. Lett. 2000, 85, 1710-1713. (b) Ulbricht, H.; Moos, G.; Hertel, T. Phys. ReV. B 2002, 66, 075404.

(17) The progress of the on-chip biotinylation reaction can be monitored by measuring Isd(Vg). Biotin-N-hydroxysuccinimide ester reacts readily with primary amines in PEI under ambient conditions, thus reducing the electron donating of PEI. However, after 1 h the yield of the reaction is only _{—}75%; several hours are required to complete the reaction. The device characteristic at different stages of the biotinylation reaction is presented in Supporting Information.

(18) Wilchek, M.; Bayer, E. A. Methods Enzymol. 1990, 184, 49.

(19) Cumings, J.; Star, A.; Gabriel, J.-C.; Bradley, K.; Gru\ddot{}ner, G. “Influence of Mobile Ions on Nanotube Based FET Devices”, submitted for publication.

### Example 2

The affinity of proteins to nanotubes was explored by using multiwall nanotubes and also a network of bundles of nanotubes, both the multiwall nanotubes and the bundles having a diameter similar to the diameter of the protein in question. Nanotubes (2 mg) dispersed in were incubated with BSA-dye conjugate (1 mg/ml, 1.25 microgram) overnight, were washed with PBS. Depletion of BSA from the solution was evaluated using UV absorption (580 nm) of fluorescent dye. Significant depletion was observed, however the amount of attached protein per nanotube was be evaluated as the surface area of the bundles was not measured. SEM images indicated significant coverage, as observed by other groups.

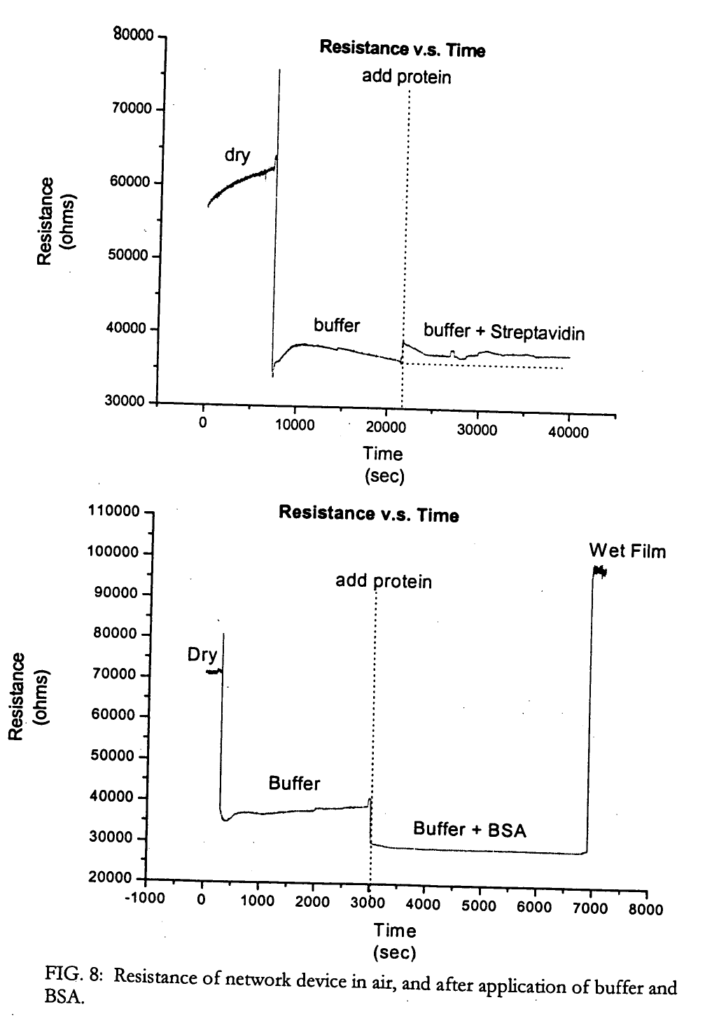

When adding proteins (BSA) the resistance drops, as illustrated on FIG. 8. This is because the BSA solution applied is 4 times more conductive than the buffer solution. When calculating what value of resistance the film has (considering the network as a resistance in parallel with buffer) it is roughly 30% higher than the value of the resistance in buffer without the protein added. This can also be seen at the end of the data run when the fluid is removed from the film. The resistance increases above what the dry value of resistance of the film.

The data were analyzed by solving a set of linear equations which related the networks resistance at each point to the film's resistance when the film is initially submerged in the fluid (during “Buffer” section on graph). This is done using the relationship between the conductivity of the buffer and the conductivity of the protein solution.

$$\frac{- \frac{1}{Rf\_prot} + \frac{1}{Rtot\_prot}}{- \frac{1}{Rf\_buf} + \frac{1}{Rtot\_buf}} == \frac{Lprot}{Lbuf}$$

Where the left-hand-side of the equation is just Rbuf/Rprot, and

Lbuf=conductivity of buffer solution

Lprot=conductivity of protein solution

Rtot_buf=total resistance measured while submerged in buffer (see “Buffer” in figure)

Rtot_prot=total resistance measured while submerged in protein (see “Protein” in figure)

Rf_buf=Film's Resistance in Buffer(this is the initial “guess”, based on “Dry”)

Rf_prot=Film's Resistance in Protein (solve for in equation above)

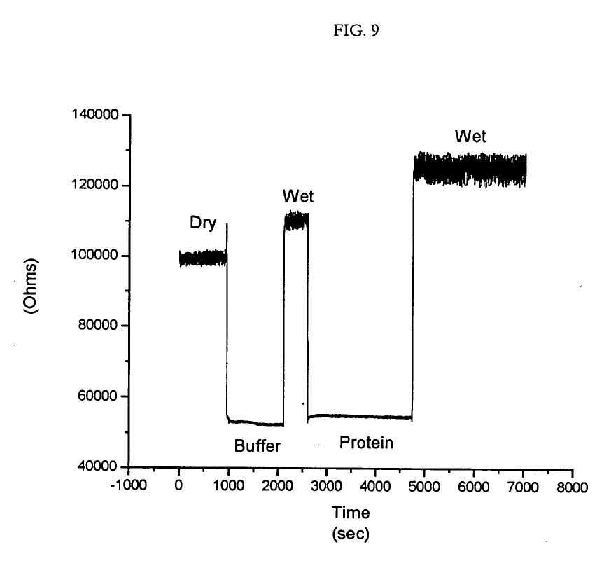

A simpler analysis can be made where the values of the film's resistance are assumed to correspond to the values measured on the “wet” section of the graph (see FIG. 9). This however leads to inconsistencies when compared to the measurements of the conductivities of the buffer/protein solutions. Therefore, the more detailed analysis is preferred, and is described below.

Data:

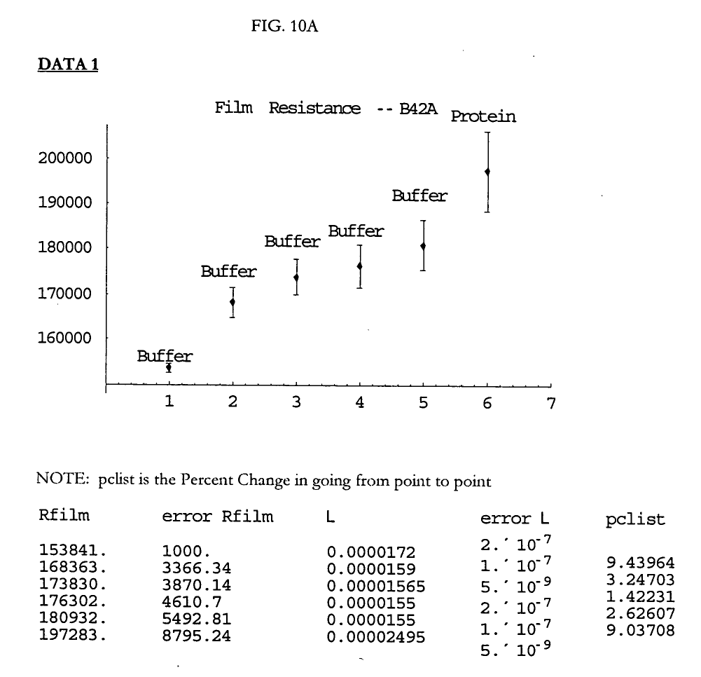

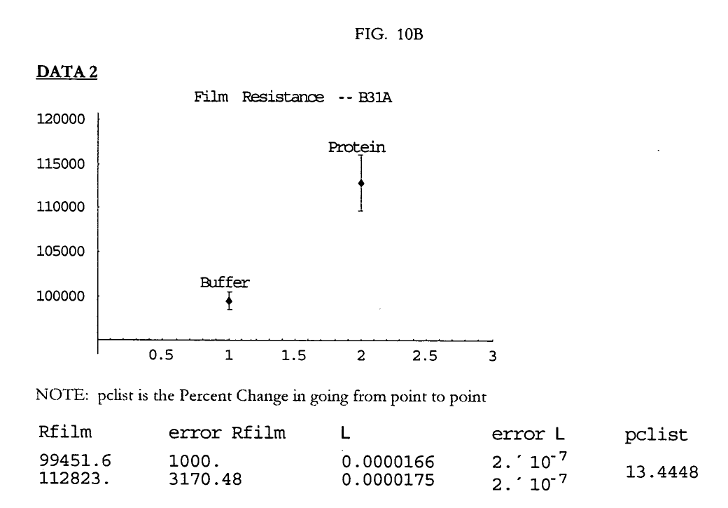

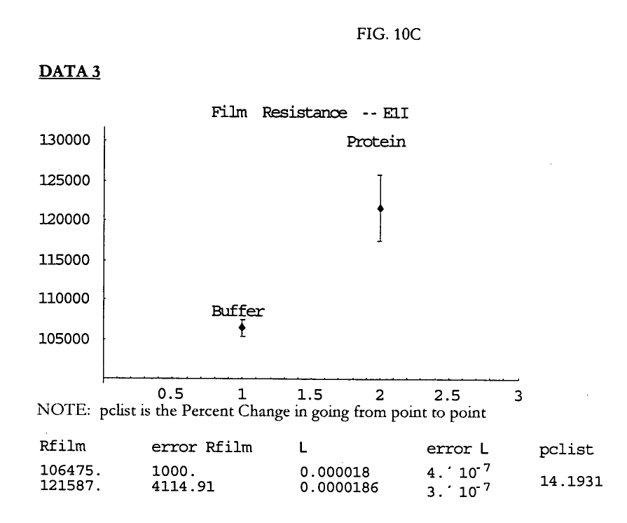

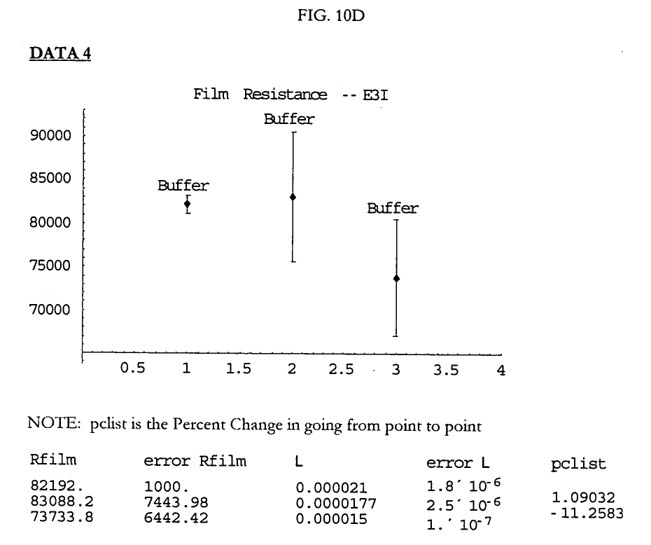

The data below show the film's resistance. There are 2 different cases:

| | |
|---|---|
| 1) Add Buffer, Add Protein | (3 data sets, FIGS. 10A-10C) |
| 2) Add Buffer, Add Buffer | (1 data set, FIG. 10D) |

2 different protein concentrations were used. For Data 1 (FIG. 10A), BSA of approximately 1.3 micro-molar (micro Moles/Liter) was used. For Data 2 and 3 (FIGS. 10B and 10C), BSA of approximately 13 micro-molar was used. The buffer used was PBS of approximately 5 micro-molar concentration.

### Conclusion

In all the data sets where protein is added, the resistance of the film increases by ˜10%. In the first data set the network was washed with buffer many times before adding the proteins. In this data set (FIG. 10A) the film's resistance increases by 9% when the buffer is added, and then much less each time more buffer is added (˜3%). This latter increase is most likely due to tubes being washed away, and is seen to diminish as we make more replacements of the buffer. When the protein solution is added, there is a 9% jump in the films resistance. This is a strong case for having a noticeable change upon addition of proteins. Taking error bars into account, there is still a ˜1.1% increase in the networks resistance.

In conclusion, nanotube networks offer appropriate alternatives to detection of proteins. Such networks could also be functionalized for binding specificity, using strategies similar to those explored using nanotube based FET devices discussed in Example 1.

Throughout this application, various publications are referenced (articles such as Snow et al., Appl. Phys. Lett. 82(13): 2145-2147 (2003) and Chen et al., PNAS 100: 4984-4989 (2003); patents, patent applications etc.). The disclosures of these publications are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. A method for detecting an interaction between a putative binding partner and a biomolecule coupled to a semiconductor nanostructured material having measurable level of resistance, the method comprising:
measuring the level of resistance of semiconductor nanostructured material coupled to the biomolecule in the presence of a medium without a putative binding partner;
exposing the semiconductor nanostructured material coupled to the biomolecule to a medium having a putative a binding partner;
measuring the level of resistance of semiconductor nanostructured material coupled to the biomolecule in the presence of a medium having a putative a binding partner;
wherein the level of resistance of so measured is modulated by the binding status of biomolecule such that a change in the level of resistance so measured provides an indication of an interaction between the binding partner and the biomolecule.

2. A method for detecting a conformational change in a biomolecule coupled to a semiconductor nanostructured material having measurable level of resistance, the method comprising:
measuring the level of resistance of semiconductor nanostructured material coupled to the biomolecule in a first conformation;
exposing the semiconductor nanostructured material coupled to the biomolecule to a medium having molecules which may induce a conformational change in the biomolecule;
measuring the level of resistance of semiconductor nanostructured material coupled to the biomolecule in the presence of the medium having molecules which may induce a conformational change in the biomolecule;
wherein the level of resistance of so measured is modulated by the conformational state of biomolecules such that a change in the level of resistance so measured provides an indication of a change in the conformation of the biomolecule.
